(19) United States
(12) Patent Application Publication
(10) Pub. No.: US 2016/0270968 A1
(43) Pub. Date: Sep. 22, 2016

(54) TWO-PART BANDAGE WITH REPLACEABLE WOUND COVERING PORTION

(71) Applicants: Amanda J. Stanford, Northville, MI (US); Madison D. Stanford, Northville, MI (US)

(72) Inventors: Amanda J. Stanford, Northville, MI (US); Madison D. Stanford, Northville, MI (US)

(21) Appl. No.: 14/664,716

(22) Filed: Mar. 20, 2015

(51) Int. Cl. A61F 13/02; A61F 13/00

(52) U.S. Cl. CPC A61F 13/0236; A61F 13/00063; A61F 2013/00153; A61F 2013/00182

(57) ABSTRACT

A two-piece bandage is disclosed for treating wound on a patient. The bandage includes a boundary portion including a bottom surface comprising adhesive for attaching the boundary portion to skin of a patient, a top surface, and an open window in the middle of the boundary portion permitting one to view a wound through the open window. The bandage further includes a plurality of replaceable wound covering portions, each replaceable wound covering portion comprising a padded section comprising a material to dress the wound. Each replaceable wound covering portion can be sequentially positioned with the padded section proximate to the wound and adhered to the boundary portion.

### TECHNICAL FIELD

This disclosure is related to an bandage used in medical care. In particular, the disclosure is related to a two-part bandage including a first boundary portion adhered to the skin of the patient and including a window and a second wound covering portion covering the window and adhering to the boundary portion.

### BACKGROUND

The statements in this section merely provide background information related to the present disclosure. Accordingly, such statements are not intended to constitute an admission of prior art.

Adhesive bandages are used to cover a wound on a patient. A known adhesive bandage includes a portion of the bandage with an adhesive safe for contact with human skin applied thereto. The bandage also includes a portion of the bandage with a fabric or other material intended to rest upon or proximate to a wound on the patient's skin.

Removal of the bandage or checking or cleaning the wound requires one to peel the adhered bandage off the skin of the patient. Some bandages include relatively strong adhesives and are firmly affixed to the skin of the patient. Removing such bandages can be painful or can irritate the skin of the patient. Repeated application and removal of such bandages can damage the skin over time. Other bandages include relatively weak adhesives, reducing pain and irritation caused by removing the bandage, but also increasing a likelihood that the bandage will accidentally fall off or adhere poorly and fail to adequately protect the wound.

### SUMMARY

A two-piece bandage is disclosed for treating wound on a patient. The bandage includes a boundary portion including a bottom surface comprising adhesive for attaching the boundary portion to skin of a patient, a top surface, and an open window in the middle of the boundary portion permitting one to view a wound through the open window. The bandage further includes a plurality of replaceable wound covering portions, each replaceable wound covering portion comprising a padded section comprising a material to dress the wound. Each replaceable wound covering portion can be sequentially positioned with the padded section proximate to the wound and adhered to the boundary portion.

### BRIEF DESCRIPTION OF THE DRAWINGS

One or more embodiments will now be described, by way of example, with reference to the accompanying drawings, in which:

FIG. 1 illustrates an exemplary two-part bandage including a first boundary portion and a second replaceable wound covering portion, in accordance with the present disclosure;

FIG. 2 illustrates a sheet of bandage components that can be supplied or manufactured to include a boundary portion of a two-part bandage and a plurality of replacement wound covering portions, in accordance with the present disclosure;

FIG. 3 illustrates exemplary packaging of a two-part bandage providing for individual sterile packaging of each of the components of the bandage and for retention of the packaging close to the patient, in accordance with the present disclosure;

FIG. 4 illustrates an exemplary two-part bandage configured to be applied to a long narrow wound such as a surgical incision, in accordance with the present disclosure;

FIG. 5 illustrates an exemplary two-part bandage configured to be applied around a patient's eye, in accordance with the present disclosure;

FIG. 6 illustrates an exemplary bottom side of a replaceable wound covering portion, in accordance with the present disclosure;

FIG. 7 illustrates an exemplary replaceable wound covering portion in profile including a thin padded section, in accordance with the present disclosure;

FIG. 8 illustrates another exemplary replaceable wound covering portion in profile including a thick padded section, in accordance with the present disclosure;

FIG. 9 illustrates and exemplary two-part bandage including a plurality of replaceable wound covering portions, each of the wound covering portions including different graphic patterns upon a top surface, in accordance with the present disclosure;

FIG. 10 illustrates an exemplary bandage for use on a knee or elbow including the disclosed wound covering portion, in accordance with the present disclosure;

FIG. 11 illustrates an exemplary boundary portion of a bandage for use on a intravenous injection site or mole removal site, in accordance with the present disclosure;

FIG. 12 illustrates an exemplary boundary portion of a bandage for use on a knuckle, elbow, or knee, in accordance with the present disclosure;

FIG. 13 illustrates an additional exemplary boundary portion of a bandage for use on a knuckle, elbow, or knee, in accordance with the present disclosure;

FIG. 14 illustrates an exemplary boundary portion of a bandage for use on a fingertip, in accordance with the present disclosure;

FIG. 15 illustrates an exemplary boundary portion of a bandage for use on a knuckle, in accordance with the present disclosure;

FIG. 16 illustrates an exemplary boundary portion of a bandage for use on a shoulder, arm pit, or knee, in accordance with the present disclosure; and

FIG. 17 illustrates an exemplary bandage configured to be applied to an incision wound, for example, a surgical site, in accordance with the present disclosure.

### DETAILED DESCRIPTION

A two-part bandage is disclosed including a first boundary portion adhered to the skin of the patient and including an open window and a second wound covering portion covering the window and adhering to the boundary portion. A removable, replaceable wound covering portion permits one to dress or treat a wound repeatedly during the healing of the wound without repeatedly pulling the bandage off of the skin of the patient. Further, by using a new wound covering portion each time the portion is removed, sterility of the dressing is improved over a dressing that is simply reapplied to the wound.

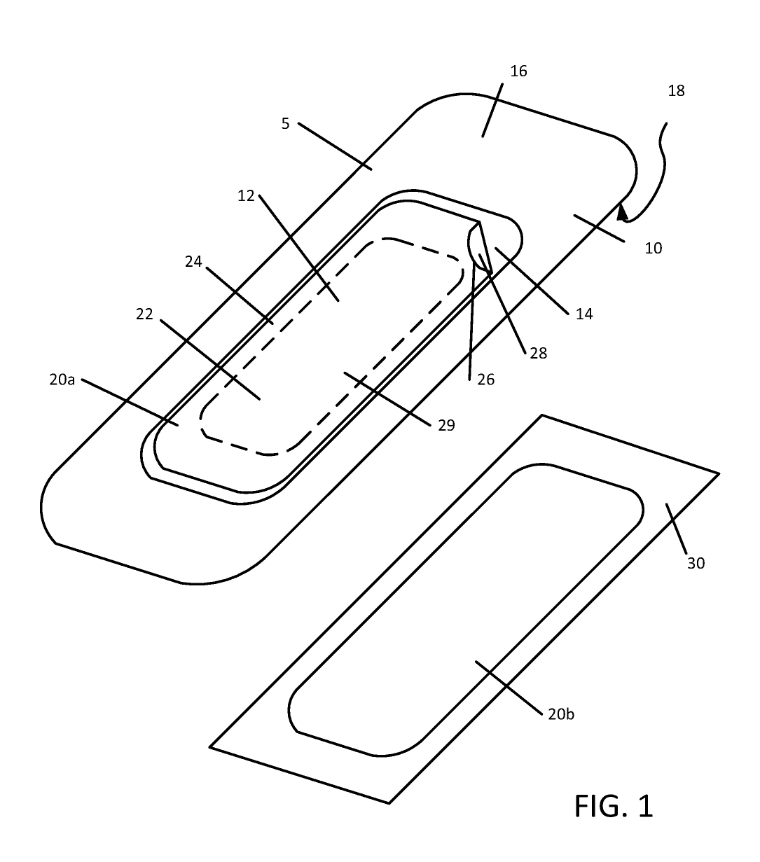

Referring now to the drawings, wherein the showings are for the purpose of illustrating certain exemplary embodiments only and not for the purpose of limiting the same, FIG. 1 illustrates an exemplary two-part bandage including a first boundary portion and a second replaceable wound covering portion. Two-part bandage 5 is illustrated including a first boundary portion 10 and a second wound covering portion 20a. Boundary portion 10 includes a polymerized or cloth based bandage material configured to be adhered to the skin of a patient. Boundary portion 10 has a top surface 16 and a bottom surface 18. Bottom surface 18 includes adhesive configured to temporarily stick to the skin of the patient. The adhesive can include any adhesive used in medical devices known in the art. Boundary portion 10 is configured to be adhered once to the skin of the patient and remain there as long as the patient needs a bandage. Boundary portion 10 includes open window 12, illustrated in FIG. 1 by a dotted line. Open window 12 can be situated over a wound, such that the bottom surface 18 of boundary portion 10 does not come into contact with the wound. Different two-part bandages with different shapes and sizes of open windows can be provided for different wounds. In one exemplary embodiment, the size of the window can be described or printed as an outline upon packaging of the bandages to aid a person providing medical care or first aid to select a correct bandage for a particular wound.

Replaceable wound covering portion 20a is illustrated adhered to top surface 16 of boundary portion 10. Replaceable wound covering portion 20a includes top surface 29 and bottom surface 28. A corner 26 of replaceable wound covering portion 20a is illustrated peeled back from boundary portion 10 to reveal a patch of bottom surface 28. An adhesive is used upon one of bottom surface 28 or patch 14 of top surface 16 to adhere replaceable wound covering portion 20a to boundary portion 10. While such adhesive can be used on either surface, adhesive on bottom surface 28 is advantageous because any fresh replaceable wound covering portion used upon the bandage would have new adhesive that would be unlikely to have been contaminated or made less effective through previous use of the bandage. Exemplary replaceable wound covering portion 20a of FIG. 1 includes a racetrack shaped adhesive coating 24 around a perimeter of bottom surface 28. Coating 24 is shaped such that none of the adhesive or only an incidental amount of adhesive contacts the skin of the patient through window 12. Replaceable wound covering portion 20a includes a padded section 22 located upon bottom surface 28 including gauze or a similar substance known in the art to be placed upon or proximate to or to dress the wound itself. Padded section 22 can be sized to be similar in size to open window 12, such that any skin visible within window 12 will be covered with the gauze material once replaceable wound covering portion 20a is attached to boundary portion 10.

Additionally, a second replaceable wound covering portion 20b is illustrated initially adhered to backing paper 30. Backing paper 30 is known in the art and can include any surface with a coating configured to temporarily hold an object with an adhesive layer and later release that object with the adhesive layer for subsequent adhesion to another object. At any point through the treatment of the patient, replaceable wound covering portion 20a can be removed from bandage 5 and replaced with replaceable wound covering portion 20b. Because replaceable wound covering portion 20a is only adhered to boundary portion 10 or is only slightly adhered to the patient's skin through incidental contact through window 12, removal of replaceable wound covering portion 20a does not irritate the skin of the patient, and a fresh gauze section can be repeatedly provided to the wound without the discomfort of having to pull the adhesive away from the skin of the patient. Boundary portion 10 is removed only at the end of the treatment period. Because boundary portion 10 is only removed once and is not removed and reapplied a number of times, a more aggressive adhesive could be used to keep boundary portion 10 in place throughout the treatment and the skin only has to endure one removal of the adhesive.

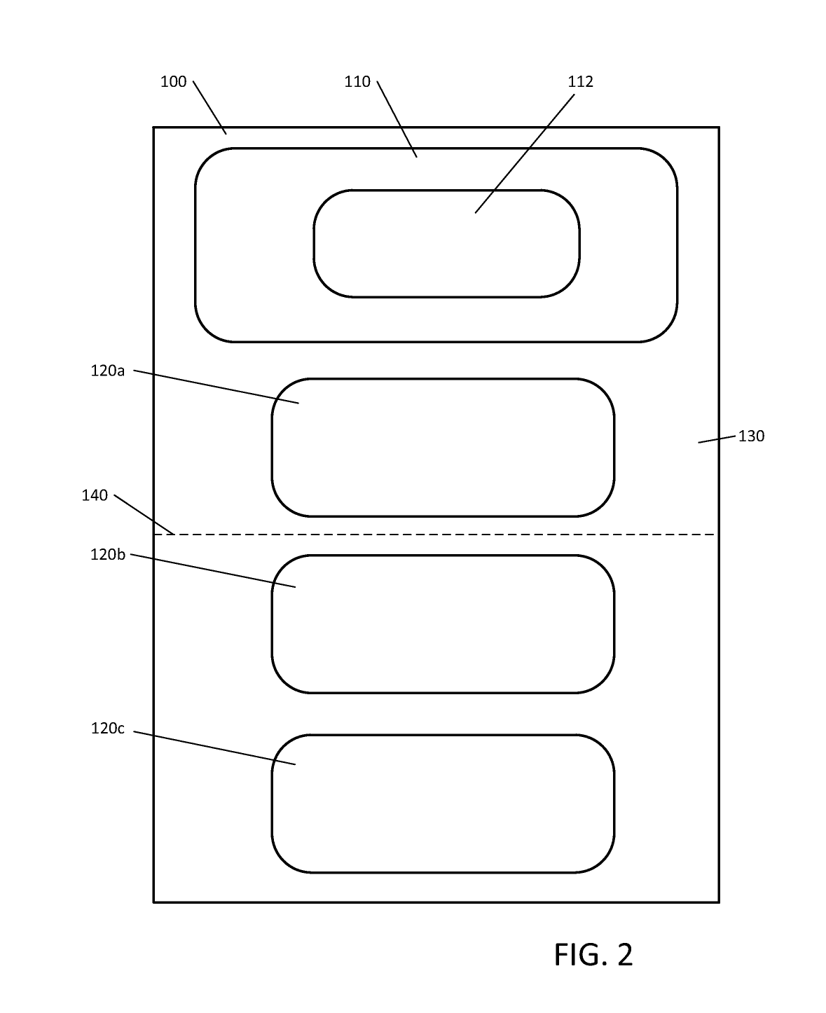

FIG. 2 illustrates a sheet of bandage components that can be supplied or manufactured to include a boundary portion of a two-part bandage and a plurality of replacement wound covering portions. Sheet 100 is illustrated including backing paper 130, boundary portion 110 and replaceable wound covering portions 120a, 120b, and 120c. Sheet 100 can be provided as a single unit, for example, within a sterile plastic or paper bag. Upon intended use, a person can rip open the package and remove boundary portion 110 from backing paper 130. Boundary portion 110 can be situated to the skin of the patient such that all of a wound on the patient is within open window 112, with adhesive attaching boundary portion 110 to the skin. The person can then place wound covering portion 120a upon boundary portion 110, with adhesive attaching the wound covering portion 120a to the boundary portion. In another embodiment, boundary portion 110 can be provided with one of the replaceable wound covering portions initially already attached to boundary portion 110. Sheet 100 can be retained with the remaining replaceable wound covering portions 120b and 120c still adhered to backing paper 130 for later use when wound covering portion 120a needs to be replaced. In one embodiment, sheet 100 can be perforated in at least one location to permit a portion of backing paper 130 to be removed from the sheet once a portion of the bandage components are removed for use. Exemplary perforation 140 is illustrated.

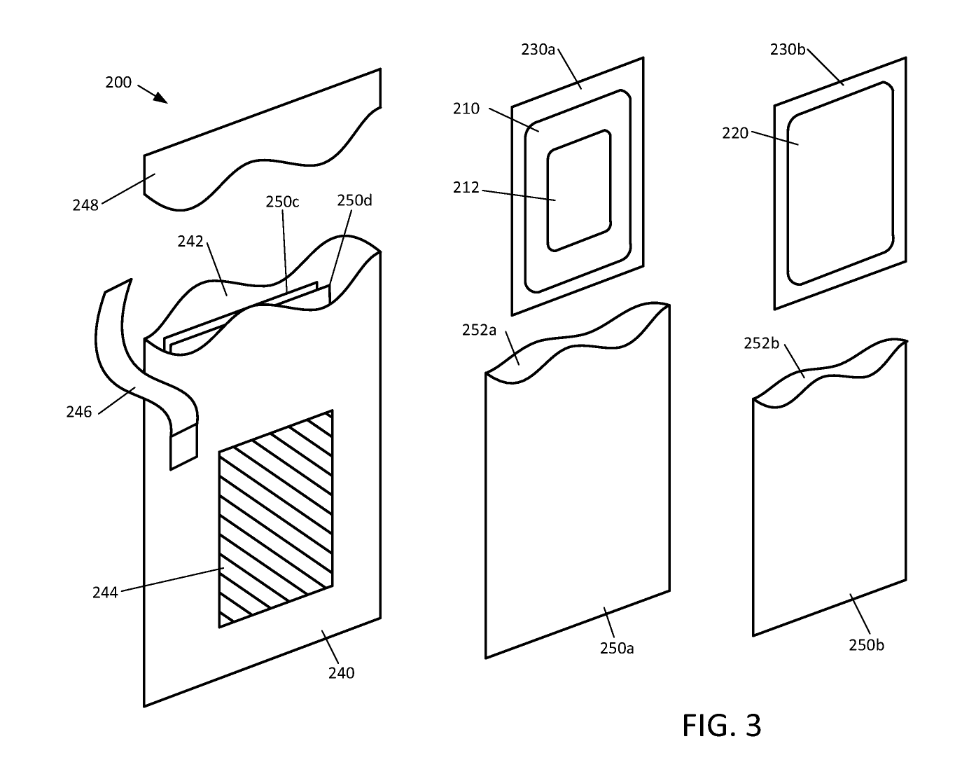

FIG. 3 illustrates exemplary packaging of a two-part bandage providing for individual sterile packaging of each of the components of the bandage and for retention of the packaging close to the patient. Two-part bandage unit 200 is illustrated including two-part bandage packaging 240. Two-part bandage packaging 240 includes a paper, plastic, or other material envelope, box, or other container that can initially hold individually wrapped bandage components. In the example of FIG. 3, packaging 240 has been torn open with removed top 248 discarded. Component packaging 250a and 250b have been removed and include paper or other similar packaging initially holding boundary portion 210 and replaceable wound covering portion 220, respectively. Packaging 250a and 250b have both been removed from packaging 240 and opened to remove boundary portion 210 and replaceable wound covering portion 220, respectively, from cavities 252a and 252b, respectively. Boundary portion 210 can subsequently be removed from backing paper 230a and situated to a wound by locating the wound to open window 212. Replaceable wound covering portion 220 can then be adhered to boundary portion 210. In another embodiment, replaceable wound covering portion 220 could be provided already attached to boundary portion 210. Component packaging 250c and 250d remain within cavity 242 of packaging 240 still retained in an unopened state and each can contain one or more replaceable wound covering portions. As the patient needs a replaceable wound covering portion replaced, one of the component packaging 250c or 250d can be removed and opened.

Packaging 240 is provided with optional adhesive section 244 and tether 246 for attaching packaging 240 to a patient's hospital bed, end table, or any other convenient location such that the packaging 250c and 250d can be accessed as needed. Adhesive section 244 can come with backing paper initially attached. Tether 246 can include adhesive, a pair of snap features, or any other mechanism known in the art for temporarily attaching a tether to an object.

In some embodiments of the disclosure, the outer perimeter of a replaceable wound covering portion can be smaller than the outer perimeter of the matching boundary portion. In the example of FIG. 3, replaceable wound covering portion 220 is illustrated to include the same size outer perimeter as boundary portion 210. This can aid in easily aligning the portions and ensuring that any gauze attached to wound covering portion 220 is properly aligned to window 212.

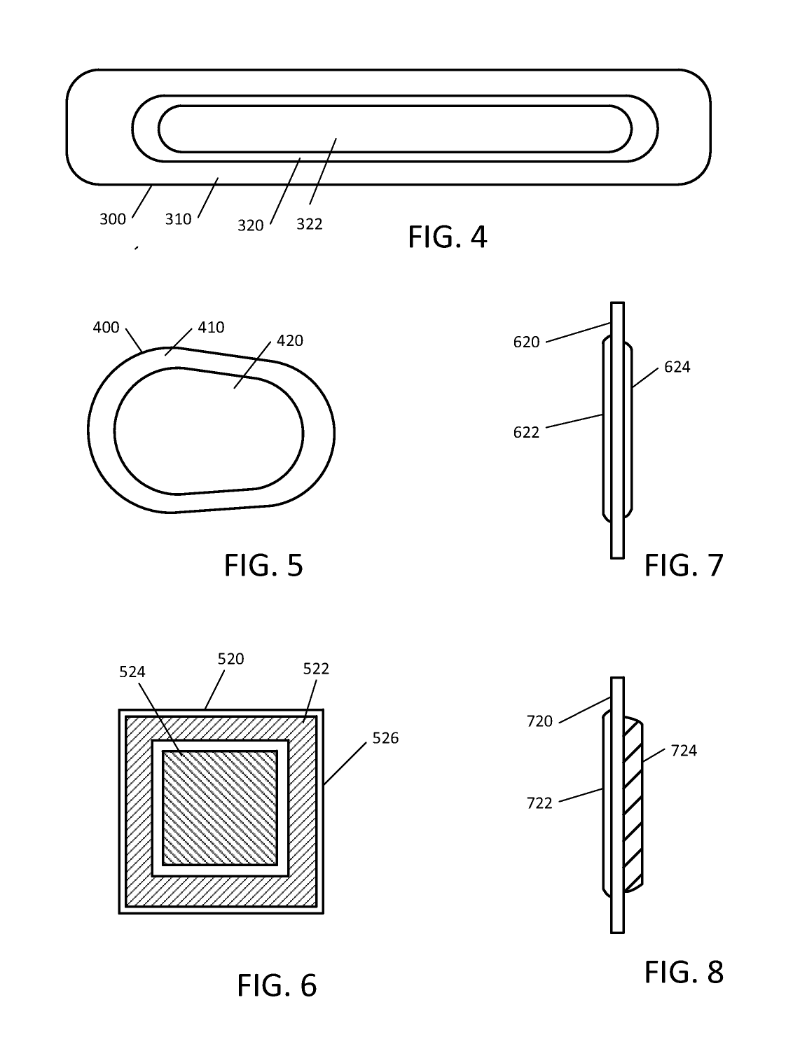

FIG. 4 illustrates an exemplary two-part bandage configured to be applied to a long narrow wound such as a surgical incision. Two-part bandage 300 is illustrated and includes boundary portion 310 and replaceable wound covering portion 320. Boundary portion 310 includes a long narrow open window to permit one to view a long incision, possibly including stitches or other medical applications to close the wound. Raised padding portion 322 is visible upon wound covering portion 320. Depending upon the thickness and rigidity of the material used for wound covering portion 320 and the thickness of the padding material used, such as gauze, the raised padding portion 322 may or may not be evident on a bandage.

FIG. 5 illustrates an exemplary two-part bandage configured to be applied around a patient's eye. Two-part bandage 400 includes boundary portion 410 and wound covering portion 420 applied thereto. Boundary portion 410 includes an open window sized to permit one to view the eye within the window. In the exemplary embodiment of FIG. 5, no raised padded section is evident due to a thicker material being used for portion 420, a thinner gauze material being used for the padding, or both. A number of different shapes and sizes of two-piece bandages are envisioned, and the disclosure is not intended to be limited to the examples provided herein.

FIG. 6 illustrates an exemplary bottom side of a replaceable wound covering portion. Replaceable wound covering portion 520 includes a square-shaped perimeter 526, a padded portion 524 to be placed proximate to a wound, and an adhesive portion 522 configured to attach the wound covering portion to a matching boundary portion. In some embodiments, because the adhesive used on adhesive portion 522 is going to be attached to a boundary portion and not to the skin of the patient, a more aggressive adhesive can be used than would be used on a directly applied bandage. In this way, adhesive portion 522 in some embodiments can be smaller or have less surface area than a typical directly applied bandage.

FIG. 7 illustrates an exemplary replaceable wound covering portion in profile. Replaceable wound covering portion 620 includes raised padding portion 622 and padded section 624. Padded section 624 can include any number of wound covering materials known in the art and can include an absorbent material, a coating or membrane configured to prevent clotted blood from sticking to the padding, medication or a treated coating, and any other materials or substances known in the art for use upon a bandage applied to a wound. FIG. 8 illustrates another exemplary replaceable wound covering portion in profile. Replaceable wound covering portion 720 includes raised padding portion 722 and padded section 724. Padded section 724 is thicker and can be more absorbent, for example, so that a medicated liquid can be applied to the bandage. In this way, timed or scheduled replacement of the wound covering portions can be used according to a treatment plan to periodically apply a medicated liquid, powder, or ointment to the wound.

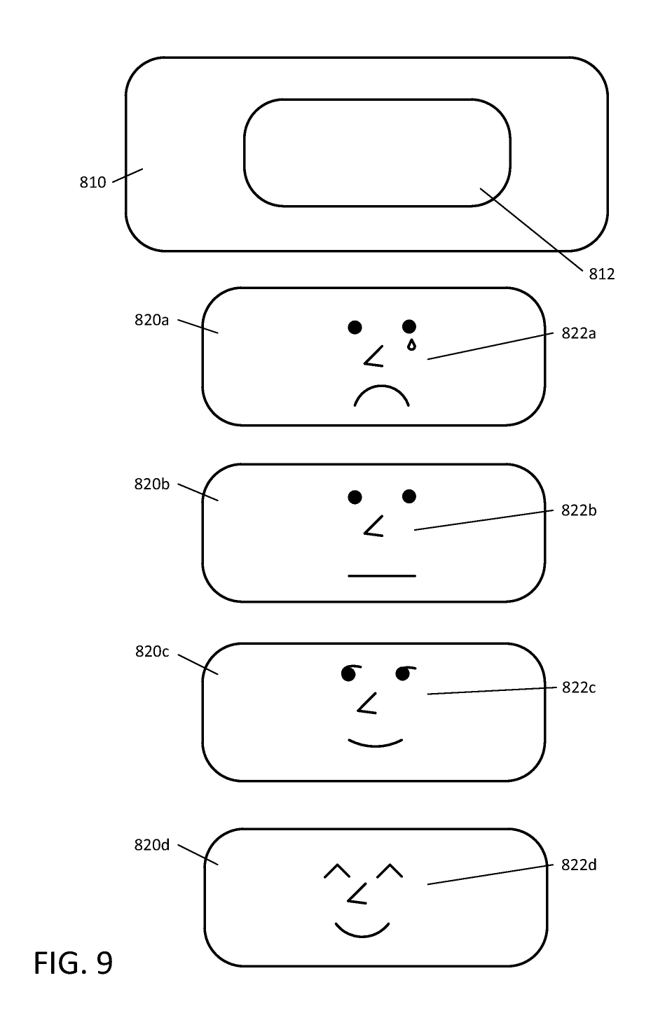

FIG. 9 illustrates and exemplary two-part bandage including a plurality of replaceable wound covering portions, each of the wound covering portions including different graphic patterns upon a top surface. Boundary portion 810 is illustrated including open window 812. Replaceable wound covering portions 820a, 820b, 820c, and 820d are illustrated, each including a respective graphic pattern 822a, 822b, 822c, and 822d imprinted thereupon. A number of exemplary graphic patterns are envisioned. For example, three different colors can be used, for example, indicating a shift in a hospital in which the wound covering portion was last replaced. In this way, a medical professional could easily audit a floor to make sure that all bandages had been updated for a particular shift. In another example, a doctor could have a range of wound covering portions to select from, each of a set of patterns, for example, including a letter designation, indicating an evaluation of how well the wound was healing or if there was any sign of infection. In the exemplary embodiment of FIG. 9, graphic patterns 822a, 822b, 822c, and 822d include a progression of facial expressions, which could be used, for example, to encourage a younger patient through a healing time, first, to sympathize with the patient after an injury or procedure, and, later, to remind the patient that happier times are on the way as the wound heals. A number of exemplary graphic patterns are envisioned, and the disclosure is not intended to be limited to the examples provided herein.

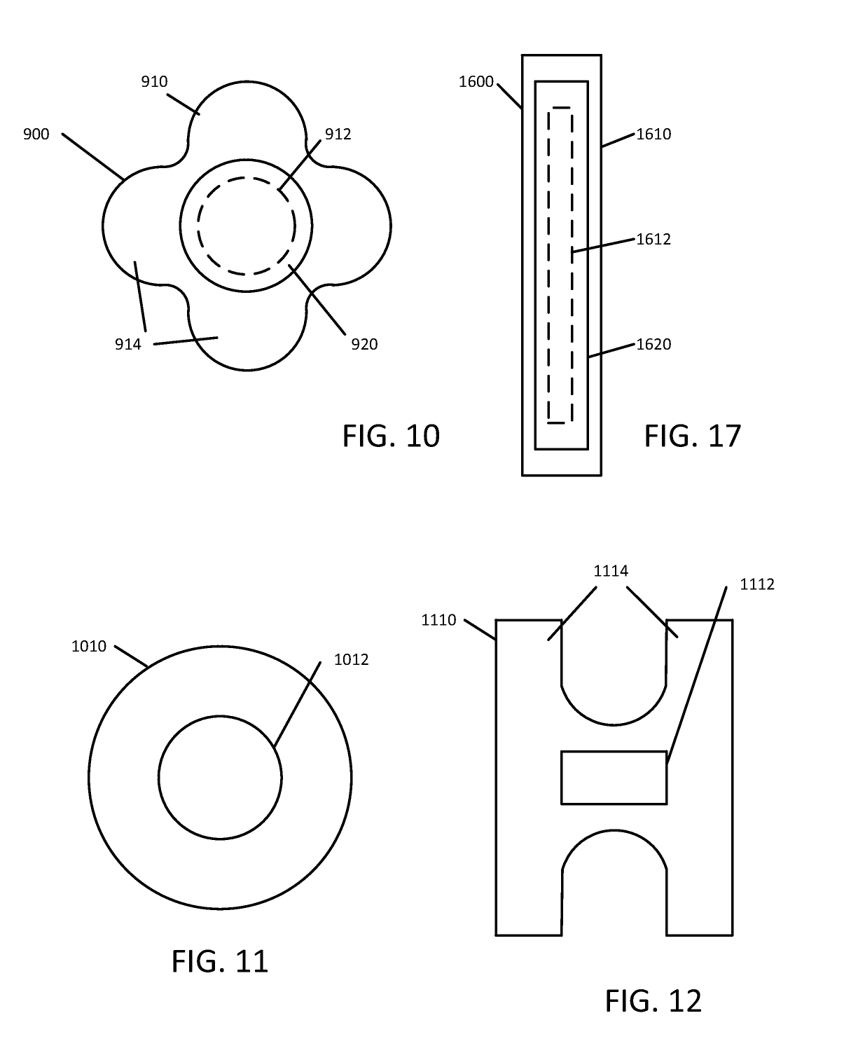

A wide variety of bandages can include boundary portions and wound covering portions as disclosed herein. FIG. 10 illustrates an exemplary bandage for use on a knee or elbow including the disclosed wound covering portion. Bandage 900 is illustrated including boundary portion 910 and wound covering portion 920 adhered thereto. Boundary portion 910 includes treatment window 912 and four wing panels 914. Wing panels 914 increase adhesive contact between boundary portion 910 and the skin of the patient, thereby increasing a likelihood that the bandage will remain in place on a articulating body part such as a knee or elbow.

FIG. 11 illustrates an exemplary boundary portion of a bandage for use on a intravenous injection site or mole removal site. Round boundary portion 1010 includes a round window 1012.

FIG. 12 illustrates an exemplary boundary portion of a bandage for use on a knuckle, elbow, or knee. H-shaped boundary portion 1110 includes window 1112 and leg portions 1114.

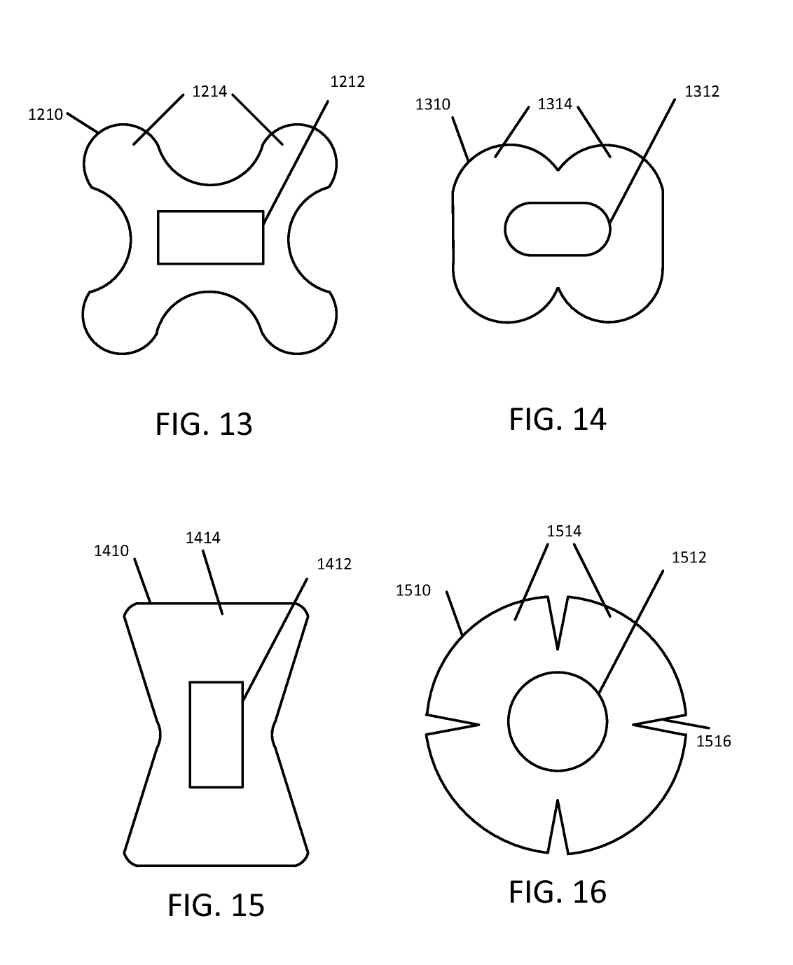

FIG. 13 illustrates an additional exemplary boundary portion of a bandage for use on a a knuckle, elbow, or knee. X-shaped boundary portion 1210 includes window 1212 and leg portions 1214.

FIG. 14 illustrates an exemplary boundary portion of a bandage for use on a fingertip. Four-lobe boundary portion 1310 includes window 1312 and four lobe portions 1314.

FIG. 15 illustrates an exemplary boundary portion of a bandage for use on a knuckle. Double-taper boundary portion 1410 includes window 1412 and widening end portions 1414.

FIG. 16 illustrates an exemplary boundary portion of a bandage for use on a shoulder, arm pit, or knee. Relief-cut round boundary portion 1510 includes window 1512, round cut extending portions 1514, and relief cuts 1516.

FIG. 17 illustrates an exemplary bandage configured to be applied to an incision wound, for example, a surgical site. Bandage 1600 is illustrated including boundary portion 1610 and wound covering portion 1620. Boundary portion 1610 includes wound treatment window 1612. In order to cover a long, thin incision wound site, the boundary portion and the corresponding window can each have high aspect ratios, with thin widths and long lengths. Non-limiting examples of boundary portions can have aspect ratios of 5:1, 10:1, or 20:1. The provided boundary portion shapes and proportions, window shapes and proportions, and wound covering portion shapes and proportions are provide as non-limiting examples, and the disclosure is not intended to be limited to the particular examples provided.

The disclosure has described certain preferred embodiments and modifications of those embodiments. Further modifications and alterations may occur to others upon reading and understanding the specification. Therefore, it is intended that the disclosure not be limited to the particular embodiment(s) disclosed as the best mode contemplated for carrying out this disclosure, but that the disclosure will include all embodiments falling within the scope of the appended claims.

1. An apparatus comprising a two-piece bandage, comprising:
a boundary portion comprising:
a bottom surface comprising adhesive for attaching the boundary portion to skin of a patient;
a top surface; and
an open window in the middle of the boundary portion permitting one to view a wound through the open window; and
a plurality of replaceable wound covering portions, each replaceable wound covering portion comprising a padded section comprising a material to dress the wound; and
wherein each replaceable wound covering portion can be sequentially positioned with the padded section proximate to the wound and adhered to the boundary portion.

2. The apparatus of claim 1, wherein each replaceable wound covering portion comprises an adhesive applied to a bottom surface of the replaceable wound covering portion configured to adhere the replaceable wound covering portion to the boundary portion.

3. The apparatus of claim 1, wherein the plurality of replaceable wound covering portions each include different printing upon a top surface of the replaceable wound covering portion.

4. The apparatus of claim 3, wherein the different printing upon the replaceable wound covering portions include a plurality of different colors.

5. The apparatus of claim 3, wherein the different printing upon the replaceable wound covering portions include an indication to a particular hospital shift upon the printing.

6. The apparatus of claim 3, wherein the different printing upon the replaceable wound covering portions include a first printing including an unhappy face logo and a second printing including a happy face logo.

7. The apparatus of claim 1, wherein further comprising single packaging unit initially holding the boundary portion and the plurality of replaceable wound covering portions.

8. The apparatus of claim 7, wherein the single packaging unit comprises a affixing mechanism to affix the single packaging unit proximate to the patient.

9. The apparatus of claim 8, wherein the affixing mechanism comprises an adhesive patch.

10. The apparatus of claim 8, wherein the affixing mechanism comprises a tether.

11. The apparatus of claim 1, wherein one of the replaceable wound covering portions is provided within packaging initially adhered to the boundary portion.

12. The apparatus of claim 1, wherein the boundary portion is initially provided within packaging with the open window uncovered permitting one to view the wound while the boundary portion is positioned.

13. The apparatus of claim 1, wherein at least one of the replaceable wound covering portions is provided within individual packaging.

14. The apparatus of claim 1, wherein a portion of the plurality of replaceable wound covering portions is provided on a single piece of backing paper.

15. The apparatus of claim 1, wherein the boundary portion is configured to be fitted around an eye of the patient.

16. The apparatus of claim 1, wherein the boundary portion is configured to be placed upon a knee of the patient.

17. The apparatus of claim 1, wherein the boundary portion is configured to be placed upon a knuckle of the patient.

18. The apparatus of claim 1, wherein the boundary portion is configured to be placed upon an incision wound site of the patient.

19. The apparatus of claim 18, wherein the boundary portion includes a length to width ratio of at least five to one.

20. The apparatus of claim 1, wherein the padded portions of the replaceable wound covering portions comprises medication applied thereto.
